(19) United States
(12) Patent Application Publication
(10) Pub. No.: US 2026/0176550 A1
(43) Pub. Date: Jun. 25, 2026

(54) FRAGRANCE COMPOSITION AND CONSUMER PRODUCT

(71) Applicant: TAKASAGO INTERNATIONAL CORPORATION, Tokyo (JP)

(72) Inventors: Kenji TANUMA, Kanagawa (JP); Kent H. LOMBARD, New York, NY (US); Masaya OTAKE, Kanagawa (JP)

(73) Assignee: TAKASAGO INTERNATIONAL CORPORATION, Tokyo (JP)

(21) Appl. No.: 19/126,447

(22) PCT Filed: Oct. 31, 2023
(86) PCT No.: PCT/JP2023/039358

§ 371 Date: May 1, 2025

Related U.S. Application Data
(60) Provisional application No. 63/421,783, filed on Nov. 2, 2022.

(51) Int. Cl. C11B 9/00

(52) U.S. Cl. CPC C11B 9/0076

(57) ABSTRACT

The present invention relates to a fragrance composition containing a compound represented by the following general formula (1). (In the formula (1), R1 represents a hydrogen atom, a methyl group, or an ethyl group, and R2 represents a hydroxy group, an alkyl group having 1 to 5 carbon atoms, or an alkoxy group having 1 to 5 carbon atoms. The dotted line represents a single bond or a double bond. In a case where the dotted line is a single bond, X represents a methylene group or a carbonyl group. In a case where the dotted line is a double bond, X represents C—R3 (R3 represents a hydrogen atom, a methyl group, or an ethyl group, where R3 represents a methyl group or an ethyl group in a case where R1 is a methyl group).)

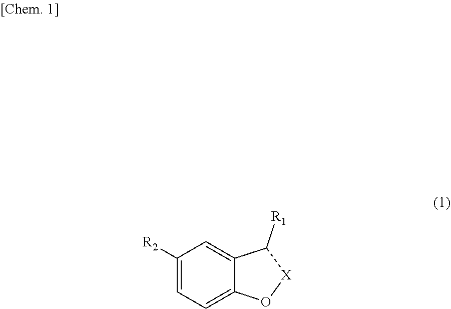

### TECHNICAL FIELD

The present invention relates to a fragrance composition containing a compound having a (dihydro)benzofuran ring, and a consumer product.

### BACKGROUND ART

In people's life currently, a variety of products with preferred scents are being sold on the market. However, in response to the increasingly diverse needs for fragranced products, there is a demand for scents having high-preference scents, and many compounds have been developed.

### SUMMARY OF INVENTION

### Technical Problem

On the other hand, since each developed compound has various characteristics in terms of scent or the like, compounds with different aroma notes are required depending on the purpose of use.

### Solution to Problem

The inventors of the present invention have found that a compound having a specific (dihydro)benzofuran ring has a unique scent such as leather-like scent or moss-like scent, and have thus completed the present invention.

The present invention relates to the following fragrance composition and the like.

A fragrance composition containing a compound represented by the following general formula (1).

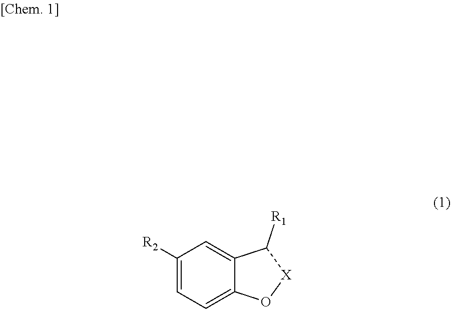

(In the formula (1), R1 represents a hydrogen atom, a methyl group, or an ethyl group, and R2 represents a hydroxy group, an alkyl group having 1 to 5 carbon atoms, or an alkoxy group having 1 to 5 carbon atoms. The dotted line represents a single bond or a double bond. In a case where the dotted line is a single bond, X represents a methylene group or a carbonyl group. In a case where the dotted line is a double bond, X represents C—R3 (R3 represents a hydrogen atom, a methyl group, or an ethyl group, where R3 represents a methyl group or an ethyl group in a case where R1 is a methyl group).)

### Advantageous Effects of Invention

By adding the compound according to the present invention, a fragrance composition having a unique scent such as leather-like scent or moss-like scent can be obtained. This composition is used in a wide range of consumer products as a fragranced composition such as a fragrance, fragrances or cosmetics, or health and hygiene products.

### DESCRIPTION OF EMBODIMENTS

Hereinafter, the present invention will be described in detail.

In the present description, a fragrance composition according to an embodiment of the present invention is also referred to as “the fragrance composition according to the present embodiment”.

In the present description, a compound represented by the formula (1a) is referred to as a “compound (1a)”. The same applies to other compounds.

The fragrance composition according to the present embodiment is a fragrance composition containing a compound represented by the following general formula (1) as an effective component. The compound (1) having a specific (dihydro)benzofuran ring has a unique scent such as leather-like scent, moss-like scent, anise-like scent, or basil-like scent.

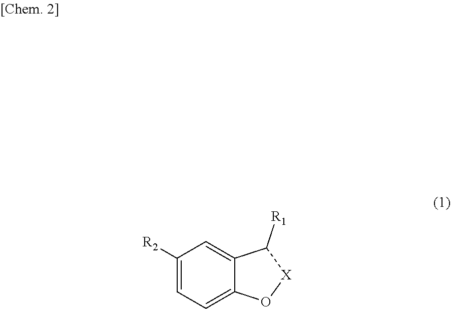

(In the formula (1), R1 represents a hydrogen atom, a methyl group, or an ethyl group, and R2 represents a hydroxy group, an alkyl group having 1 to 5 carbon atoms, or an alkoxy group having 1 to 5 carbon atoms. The dotted line represents a single bond or a double bond. In a case where the dotted line is a single bond, X represents a methylene group or a carbonyl group. In a case where the dotted line is a double bond, X represents C—R3 (R3 represents a hydrogen atom, a methyl group, or an ethyl group, where R3 represents a methyl group or an ethyl group in a case where R1 is a methyl group).)

R1 is a hydrogen atom, a methyl group, or an ethyl group. When the substituent R1 at the 3-position on the benzofuran ring is a hydrogen atom or an alkyl group having an appropriate number of carbon atoms, it is possible to obtain the compound (1) having a unique scent such as leather-like scent, moss-like scent, anise-like scent, or basil-like scent, and having a high scent intensity.

As the alkyl group having 1 to 5 carbon atoms in R2 in the general formula (1), examples thereof include a methyl group, an ethyl group, a propyl group, an isopropyl group, a butyl group, an isobutyl group, a s-butyl group, a t-butyl group, and a pentyl group. In addition, examples of the alkoxy group having 1 to 5 carbon atoms include a methoxy group, an ethoxy group, a propoxy group, an isopropoxy group, a butoxy group, an isobutoxy group, a s-butoxy group, a t-butoxy group, and a pentyloxy group.

Among them, preferred R2 is a hydroxy group, a methyl group, an ethyl group, or a propyl group.

Among the compounds represented by the general formula (1), particularly preferred compounds are as follows.

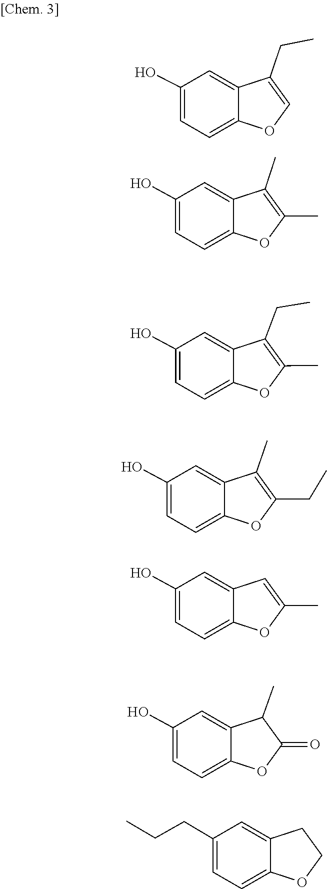

A content of the compound represented by the general formula (1) in the fragrance composition according to the present embodiment is determined depending on the blending, and is preferably 0.00001 mass % to 30 mass %, more preferably 0.01 mass % to 20 mass %, and still more preferably 0.01 mass % to 10 mass %.

The compound represented by the general formula (1) in the fragrance composition according to the present embodiment can be synthesized by the methods described in Examples to be described later.

In addition, the fragrance composition according to the present embodiment may contain one or more other fragrance retaining agents commonly used. Examples of the other fragrance retaining agents include ethylene glycol, propylene glycol, dipropylene glycol, glycerin, hexylene glycol, benzyl benzoate, triethyl citrate, diethyl phthalate, hercolin, and medium chain fatty acid triglyceride.

The fragrance composition according to the present embodiment may contain a mixed fragrance commonly used. The fragrance composition thus obtained can impart a fresh and high-preference scent.

In addition, the fragrance composition according to the present embodiment can be blended as a scent component into a consumer product in an amount commonly used in the industry to impart a unique scent and enhance the commercial value. Examples of the consumer product include a fragrance product, a cosmetic, a toiletry product, an air care product, daily necessities, household goods, an oral care product, a detergent, a softener, and a quasi-drug.

Examples of the fragrance product include perfumes, eau de parfum, eau de toilette, and eau de cologne.

Examples of the cosmetic include: basic cosmetics such as facial cleansing cream, vanishing cream, cleansing cream, cold cream, massage cream, emulsion, lotion, serum, pack, and a makeup remover; make-up cosmetics such as foundation and lipstick; hair cosmetics such as a hair tonic, a hair liquid, and a hair spray; sunscreen cosmetics such as a suntan product and a sunscreen product; and medicated cosmetics such as an antiperspirant, a body deodorant, an aftershave lotion and gel, a permanent waving agent, a medicated soap, a medicated shampoo, and a medicated skin cosmetic.

Examples of the toiletry product include: hair care products such as a shampoo, a rinse, a rinse-in-shampoo, a conditioner, a treatment, and a hair pack; skin care products such as lip balm and hand cream; soaps such as a cosmetic soap and a bath soap; body care products such as a body soap, a body shampoo, and a hand soap; and bath agents such as a bath additive (a bath salt, a bath tablet, a bath liquid, etc.), a foam bath (a bubble bath, etc.), bath oils (a bath perfume, a bath capsule, etc.), a milk bath, a bath jelly, and a bath cube.

Examples of the air care product include an aerosol, a spray, and a powder spray.

Examples of the daily necessities include a deodorant/fragrant agent.

Examples of the household goods include tissue paper and toilet paper.

Examples of the oral care product include a toothpaste, a mouthwash, a mouth spray, and gargle.

Examples of the detergent include a heavy laundry detergent, a light laundry detergent, a liquid detergent, a laundry soap, a compact detergent, and a powder soap. Examples of a fabric softener include a liquid softener and a dryer sheet. Examples of a cleaner include a cleanser, a furniture care product, a house cleaner, a toilet cleaner, a bathroom cleaner, a glass cleaner, a mold remover, and a drain cleaner. Examples of a kitchen detergent include a kitchen soap, a synthetic kitchen soap, a dish detergent, and a dishwasher detergent. Examples of a bleach include an oxidizing bleach (a chlorine-based bleach, an oxygen-based bleach, etc.), a reducing bleach (a sulfur-based bleach, etc.), and an optical bleach. Examples of the air care product include an aerosol (a spray type aerosol and a powder spray aerosol), a candle, a wax melt, a car freshener, and a free-standing, spray-type or plug-in deodorant/fragrant agent (in a solid form, gel form, liquid form, etc.). Examples of a pet care product include a pet deodorant, a pet toilet deodorant, a pet shampoo, and a pet conditioner. Examples of the household goods include various forms of tissue paper, toilet paper, a disinfectant wipe, a disinfectant spray, and the like.

Examples of the quasi-drug include a poultice and an ointment.

In the case where the fragrance composition according to the present embodiment is used in the above product, the fragrance composition according to the present embodiment is used as it is, or used in any desired state selected depending on the purpose, such as: a liquid state dissolved in polyhydric alcohols such as alcohols, propylene glycol, or glycerin; a solubilized state or dispersed state obtained by being solubilized or dispersed using surfactants such as a nonionic surfactant, an anionic surfactant, a cationic surfactant, or an amphoteric surfactant; or a microcapsule state obtained by a treatment with an encapsulating agent.

Further, the fragrance composition according to the present embodiment may be stabilized and sustained-released by being included in an inclusion agent such as cyclodextrin. These are appropriately selected and used depending on the form of the final product, for example, a liquid form, a solid form, a powder form, a gel form, a mist form, or an aerosol form.

As described above, the present description discloses the following fragrance composition and consumer product.

[1] A fragrance composition containing a compound represented by the following general formula (1).

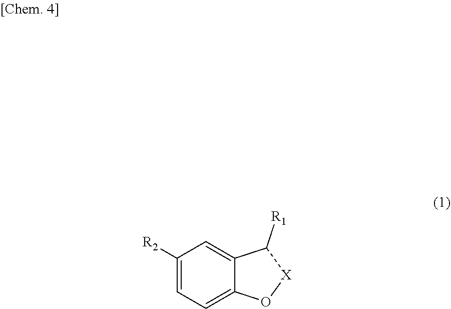

(In the formula (1), R1 represents a hydrogen atom, a methyl group, or an ethyl group, and R2 represents a hydroxy group, an alkyl group having 1 to 5 carbon atoms, or an alkoxy group having 1 to 5 carbon atoms. The dotted line represents a single bond or a double bond. In a case where the dotted line is a single bond, X represents a methylene group or a carbonyl group. In a case where the dotted line is a double bond, X represents C—R3 (R3 represents a hydrogen atom, a methyl group, or an ethyl group, where R3 represents a methyl group or an ethyl group in a case where R1 is a methyl group).)

[2] The fragrance composition according to [1], in which the compound represented by the general formula (1) includes at least one compound represented by the following formulas.

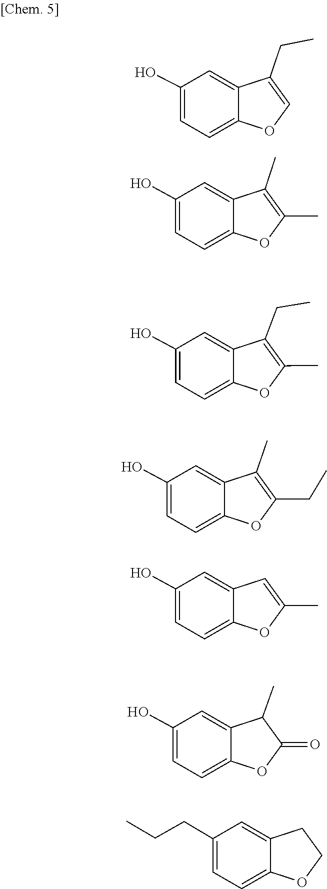

[3] A consumer product containing: the fragrance composition according to [1] or [2], in which the consumer product is selected from a fragrance product, a cosmetic, a toiletry product, an air care product, daily necessities, household goods, an oral care product, a detergent, a softener, and a quasi-drug.

### EXAMPLES

Hereinafter, the present invention will be specifically described with Examples, but the present invention is not limited to thereto in any way. The following devices were used to measure physical properties in Examples.
NMR: DRX500 (manufactured by Bruker)
GC/MS: HP5977A (manufactured by Agilent Technologies)
Column: Inertcap-1 (length: 30 m×inner diameter: 0.25 mm, film thickness: 0.25 μm) (manufactured by GL Sciences Inc.)

### (Example 1) Synthesis of 3-ethylbenzofuran-5-ol

### a) Synthesis of (E)-4-(1-buten-1-yl)morpholine

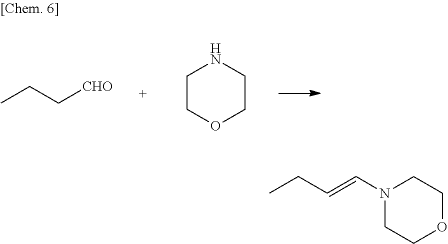

Under a nitrogen atmosphere, a mixture of 294 g (3.37 mol, 1.1 eq.) of morpholine and 166 g of potassium carbonate was warmed to 65° C. 221 g (3.06 mol) of butyraldehyde was added dropwise thereto, and after completion of the dropwise addition, the reaction was carried out at 75° C. for 2 hours. Potassium carbonate was removed by filtration through Celite, and the filtrate was subjected to Vigreux distillation, giving 271 g (yield: 63%) of (E)-4-(1-buten-1-yl) morpholine as a colorless oil, which was a fraction at 67° C. to 77° C. and 1700 Pa.

1H NMR (500 MHz, CDCl₃): δ (ppm) 6.19 (d, J=13.8 Hz, 1H), 4.87 (m, 1H), 3.98 (t, J=4.8 Hz, 4H), 2.98 (t, J=4.8 Hz, 4H), 2.53 (m, 2H), 1.52 (t, J=7.5 Hz, 3H)

13C NMR (125 MHz, CDCl₃): δ (ppm) 138.6, 104.5, 66.4, 49.3, 23.3, 15.5

### b) Synthesis of 3-ethyl-2-morpholino-2,3-dihydrobenzofuran-5-ol

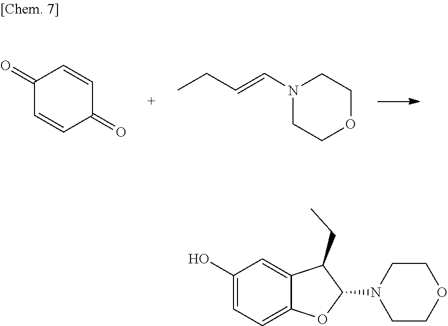

Under a nitrogen atmosphere, 1640 mL of ethyl acetate was added to 118 g (1.07 mmol) of 1,4-benzoquinone, and the mixture was cooled to 0° C. Subsequently, (E)-4-(1-buten-1-yl) morpholine (164 g, 1.05 eq) was added dropwise thereto over 45 minutes at 20° C. or lower. Thereafter, the reaction was allowed to proceed for 1 hour at room temperature. After cooling to 0° C., 12.5 g (100 mmol) of sodium sulfite was dissolved in 400 mL of water and the solution was added dropwise to the resultant of the reaction. The mixture was heated to 40° C. and the aqueous layer was removed. The organic layer was washed twice with water and then rinsed with saturated saline. The solvent was distilled off to quantitatively give 3-ethyl-2-morpholino-2,3-dihydrobenzofuran-5-ol (274 g) as a brown solid. This brown solid was used directly in the next reaction without purification.

GC/MS (EI): m/z (%) 249 (M⁺, 100), 234 (78), 220 (16), 206 (8), 190 (2), 174 (2), 162 (15), 147 (25), 121 (8), 107 (10), 86 (25), 57 (1), 41 (1)

1H NMR (500 MHz, CDCl₃): δ (ppm) 6.64 (d, J=2.2 Hz, 1H), 6.62-6.58 (m, 2H), 4.97 (d, J=3.8 Hz, 1H), 3.68 (t, J=4.7 Hz, 4H), 3.10 (q, J=3.8 Hz, 4H), 2.84-2.80 (m, 2H), 2.63-2.59 (m, 2H), 1.74-1.65 (m, 2H), 0.98 (t, J=7.4 Hz, 3H)

13C NMR (125 MHz, CDCl₃): δ (ppm) 153.4 (C), 149.3 (C), 130.4 (C), 114.7 (CH), 111.4 (CH), 108.7 (CH), 104.7 (CH), 66.9 (CH₂), 66.5 (CH₂), 46.3 (CH), 28.0 (CH₂), 10.7 (CH₃)

Scent: maritima-like

### c) Synthesis of 3-ethylbenzofuran-5-ol

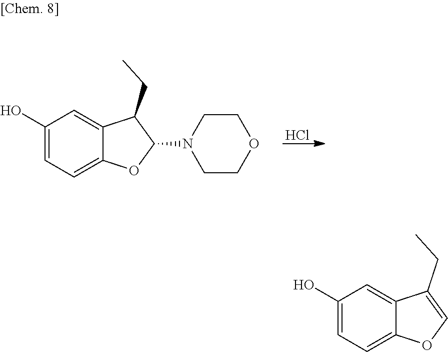 [SMILES: CC[C@@H]1c2cc(O)ccc2O[C@H]1N1CCOCC1 ; Cl ; CCc1coc2ccc(O)cc12]

Under a nitrogen atmosphere, 600 mL of acetonitrile was added to 300 g (1.2 mol) of 3-ethyl-2-morpholino-2,3-dihydrobenzofuran-5-ol, and the mixture was cooled to 0° C. 10% hydrochloric acid (1604 mL, 4.8 mol) was added dropwise thereto over 30 minutes at 10° C. or lower, and then the mixture was heated to reflux at 70° C. for 1.5 hours. The mixture was cooled to room temperature, 500 mL of toluene was added thereto, and the aqueous layer was removed. The organic layer was washed with water and rinsed with saturated saline, and then dried with magnesium sulfate. Magnesium sulfate was filtered off, and the filtrate was concentrated to give 155.3 g of crude 3-ethylbenzofuran-5-ol. This product was subjected to Vigreux distillation to give a fraction at 110° C. to 118° C. and 67 Pa, giving a crude distillate (122.8 g, yield: 75%). The crude distillate was then subjected to precision distillation in a distillation column packed with Helipack NO2 to give 114.1 g of 3-ethylbenzofuran-5-ol as a white solid (yield: 93%, from the crude distillate).

Melting point: 44° C.

GC/MS (EI): m/z (%) 162.1 (M⁺, 20), 161.1 (M⁺−1, 3), 148.1 (4), 147.1 (32), 131.1 (1), 115.1 (1), 105.1 (1), 91.1 (3), 77.1 (2), 73.6 (1), 65.1 (2), 51.1 (1), 39.0 (1)

1H NMR (500 MHz, CDCl₃): δ (ppm) 7.37 (s, 1H), 7.30 (d, J=8.7 Hz, 1H), 6.95 (d, J=2.6 Hz, 1H), 6.79 (dd, J=2.6, 8.7 Hz, 1H), 4.70-4.65 (m, 1H), 2.63 (dq, J=1.1, 7.5 Hz, 1H), 1.30 (t, J=7.5 Hz, 3H)

13C NMR (125 MHz, CDCl₃): δ (ppm) 151.1 (C), 150.5 (C), 141.7 (CH), 129.1 (C), 122.2 (C), 112.6 (CH), 111.8 (CH), 104.7 (CH), 16.9 (CH₂), 13.4 (CH₃)

Scent: leather, phenolic, animalic, cedarwood, and soft

### (Example 2) Synthesis of 2,3-dimethylbenzofuran-5-ol

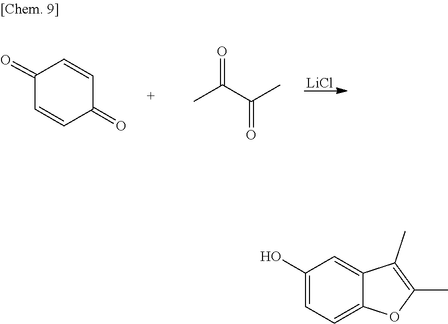

Into a 500 mL autoclave, 54.2 g (483 mmol) of 1,4-cyclohexanedione, 47.8 g (555 mmol, 1.15 eq.) of diacetyl, 150 mL of dimethylimidazole (DMI), and 20.5 g (483 mmol) of anhydrous lithium chloride were added, and the inside of the autoclave was substituted with nitrogen. The mixture was heated to an internal temperature of 160° C. and stirred for 5 hours. The mixture was cooled to room temperature, and 300 mL of ethyl acetate and 300 ml of water were added thereto. The aqueous layer was removed, the organic layer was washed with water and then rinsed with saturated saline, and then ethyl acetate was distilled off to give 187 g of a crude product (containing DMI). The crude product was subjected to Vigreux distillation to give 90 g of a crude product (purity: 80%, containing 20% of DMI) as a fraction at 120° C. to 150° C. and 67 Pa. Subsequently, in order to remove DMI, 900 mL of chloroform was added to 90 g of the crude product, followed by cooling to 0° C. Then, 235 mL (1.9 mol) of a 25% aqueous sodium hydroxide solution was added, followed by stirring for 30 minutes. The lower layer (chloroform layer) was removed, and chloroform was removed to recover 30 g of DMI. To the aqueous layer, 160 mL (12 M, 1.9 mol) of 37% hydrochloric acid was added, and then 180 mL of ethyl acetate was added. The aqueous layer was then removed, and the organic layer was washed with water. The organic layer was rinsed with saturated saline, and then ethyl acetate was distilled off. To the residue, 200 mL of hexane:ethyl acetate=1:1 was added, and the mixture was cooled to 0° C. to give 47 g of 2,3-dimethylbenzofuran-5-ol. Furthermore, the recrystallized product was subjected to bulb to bulb distillation to give a fraction at 200° C. to 220° C. and 500 Pa, giving 40 g (yield: 51%) of 2,3-dimethylbenzofuran-5-ol as a pale yellow solid.

Melting point: 78° C.

GC/MS (EI): m/z (%) 162.1 (M⁺, 29), 161.1 (M⁺−1, 19), 148.0 (2), 147.0 (23), 133.1 (6), 105.1 (1), 91.1 (2), 81.0 (1), 79.1 (1), 77.0 (2), 65.1 (1), 51.1 (1), 43.1 (1)

1H NMR (500 MHz, CDCl₃): δ (ppm) 7.19 (d, J=8.6 Hz, 1H), 6.81 (d, J=2.6 Hz, 1H), 6.69 (dd, J=2.6, 8.6 Hz, 1H), 4.68 (t, J=11.2 Hz, 1H), 2.34 (d, J=0.4 Hz, 3H), 2.09 (d, J=0.7 Hz, 3H)

13C NMR (125 MHz, CDCl₃): δ (ppm) 151.7 (C), 151.0 (C), 148.8 (C), 131.4 (C), 111.2 (CH), 110.7 (CH), 109.7 (CH), 104.0 (CH), 11.9 (CH₃), 7.91 (CH₃)

Scent: leather, animalic, moss, earthy, and woody

### (Example 3) Synthesis of 2-ethyl-3-methylbenzofuran-5-ol and 3-ethyl-2-methylbenzofuran-5-ol

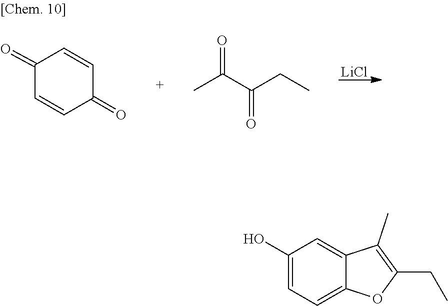

Into a 100 mL autoclave, 7 g (62.4 mmol) of 1,4-cyclohexanedione, 6.6 g (65.5 mmol, 1.05 eq.) of 2,3-pentanedione, 28 mL of DMI, and 2.6 g (62.4 mmol) of anhydrous lithium chloride were added, and the inside of the autoclave was substituted with nitrogen. The mixture was heated and stirred at 200° C. in an oil bath for 2 hours. The mixture was cooled to room temperature, and 100 mL of ethyl acetate and 100 ml of water were added thereto. The aqueous layer was removed, the organic layer was washed with water and then rinsed with saturated saline, and then ethyl acetate was distilled off to give 21 g of a crude product (containing DMI). This product was subjected to Vigreux distillation to give a fraction at 70° C. to 89° C. and 57 Pa, giving 6 g of a crude product. Further purification was performed by column chromatography, to give 4.0 g (yield: 36%) of the target product as an orange viscous oil in a ratio of 2-ethyl-3-methylbenzofuran-5-ol: 3-ethyl-2-methylbenzofuran-5-ol=55:45.

Scent: woody, leather, moss, animalic, oud, and clean

By separating 0.5 g of the product through preparative LC (two Cosmosil 5LS-2, eluent: hexane:ethyl acetate=85:15), 0.3 g of 2-ethyl-3-methylbenzofuran-5-ol and 0.2 g of 3-ethyl-2-methylbenzofuran-5-ol were isolated, respectively.

### 2-ethyl-3-methylbenzofuran-5-ol

### White Solid

GC/MS (EI): m/z (%) 176.1 (M⁺, 17), 175.1 (M⁺−1, 2), 162.1 (5), 161.1 (25), 147.1 (2), 115.1 (2), 105.1 (2), 77.1 (2), 51.1 (1)

1H NMR (500 MHz, CDCl₃): δ (ppm) 7.21 (d, J=8.6 Hz, 1H), 6.80 (d, J=2.5 Hz, 1H), 6.69 (dd, J=2.6, 8.6 Hz, 1H), 4.65-4.60 (m, 1H), 2.71 (q, J=7.6 Hz, 1H), 2.10 (s, 3H), 1.27 (t, J=7.6 Hz, 3H)

13C NMR (125 MHz, CDCl₃): δ (ppm) 156.7 (C), 151.0 (C), 148.8 (C), 131.5 (C), 111.2 (CH), 110.8 (CH), 108.7 (C), 104.1 (CH), 19.8 (CH₂), 12.7 (CH₃), 7.8 (CH₃) Scent: leather, woody, and weaker

### 3-ethyl-2-methylbenzofuran-5-ol

### White Solid

GC/MS (EI): m/z (%) 176.1 (M⁺, 16), 175.1 (M⁺−1, 1), 162.1 (4), 161.1 (29), 147.0 (2), 115.1 (1), 105.1 (1), 91.1 (1), 77.1 (1), 43.0 (1)

1H NMR (500 MHz, CDCl₃): δ (ppm) 7.20 (d, J=8.6 Hz, 1H), 6.87 (d, J=2.5 Hz, 1H), 6.68 (dd, J=2.5, 8.6 Hz, 1H), 4.67-4.62 (m, —OH, 1H), 2.56 (q, J=7.6 Hz, 2H), 2.35 (s, 3H), 1.20 (t, J=7.6 Hz, 3H)

13C NMR (125 MHz, CDCl₃): δ (ppm) 151.3 (C), 150.9 (C), 149.0 (C), 130.5 (C), 116.0 (C), 111.1 (CH), 110.8 (CH), 104.2 (CH), 16.9 (CH₂), 14.3 (CH₃), 11.9 (CH₃)

Scent: leather, moss, animalic, oud, and clean

### (Example 4) Synthesis of 2-methylbenzofuran-5-ol

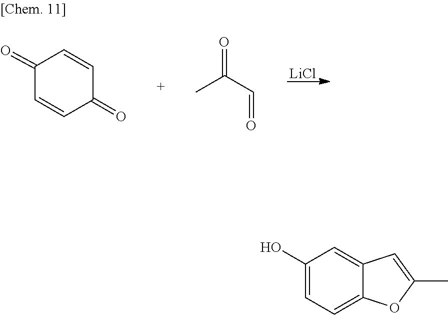

Into a 100 mL autoclave, 6 g (53.5 mmol) of 1,4-cyclohexanedione, 10.6 g (40%, 58.9 mmol, 1.1 eq.) of an aqueous methylglyoxal solution, 48 mL of DMI, and 3.4 g (62.4 mmol, 1.5 eq.) of lithium chloride were added, and the inside of the autoclave was substituted with nitrogen. The mixture was heated and stirred at 180° C. in an oil bath for 1 hour. The mixture was cooled to room temperature, and 100 mL of ethyl acetate and 100 ml of water were added thereto. The aqueous layer was removed, the organic layer was washed with water and then rinsed with saturated saline, and then ethyl acetate was distilled off to give 18 g of a crude product (containing DMI). This crude product was subjected to short path distillation to give a fraction at 100° C. to 120° C. and 65 Pa, giving 0.9 g of a crude distillate. Further purification was performed by column chromatography, to give 0.5 g (yield: 6%) of 2-methylbenzofuran-5-ol as an orange viscous oil.

1H NMR (500 MHz, CDCl₃): δ (ppm) 7.23 (d, J=8.7 Hz, 1H), 6.88 (d, J=2.6 Hz, 1H), 6.69 (dd, J=2.6, 8.7 Hz, 1H), 6.27 (s, 1H), 4.81 (br, 1H), 2.42 (s, 3H)

13C NMR (125 MHz, CDCl₃): δ (ppm) 156.6 (C), 151.3 (C), 149.8 (C), 130.1 (C), 111.4 (CH), 110.9 (CH), 105.4 (CH), 102.5 (CH), 14.2 (CH₃)

Scent: woody, moss, and powdery

### (Example 5) Synthesis of 5-hydroxy-3-methylbenzofuran-2 (3H)-one

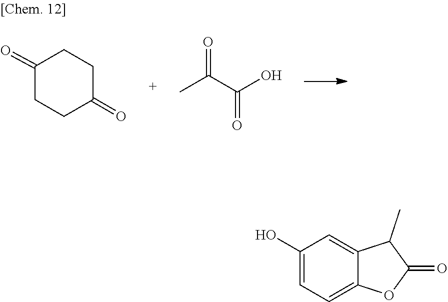

Into a 100 mL autoclave, 20.5 g (183 mmol) of 1,4-cyclohexanedione and 25 g (275 mmol, 1.5 eq.) of pyruvic acid were added, and the inside of the autoclave was substituted with nitrogen. The mixture was heated and stirred at 170° C. in an oil bath for 3 hours. The mixture was cooled to room temperature, and 100 mL of ethyl acetate and 100 ml of water were added thereto. The aqueous layer was removed, the organic layer was washed with water and then rinsed with saturated saline, and then ethyl acetate was distilled off to give 25 g of a crude product. To this, 20 mL of hexane and 10 mL of ethyl acetate were added, followed by heating to 50° C. for dissolution. After cooling to 0° C., 21 g of the generated solid was collected. Further, 20 mL of ethanol was added thereto, followed by heating to 50° C. to dissolve the solid. The mixture was then cooled to 0° C. to give the generated solid, thereby giving 5 g of 5-hydroxy-3-methylbenzofuran-2 (3H)-one (yield: 17%) as a pale yellow individual.

GC/MS (EI): m/z (%) 164.0 (M⁺, 28), 149.0 (1), 137.0 (5), 136.0 (50), 135.0 (8), 108.1 (4), 107.0 (15), 80.1 (4), 79.1 (12), 77.0 (7), 53.0 (5)

1H NMR (500 MHz, CD₃OD): δ (ppm) 6.96 (d, J=8.5 Hz, 1H), 6.77 (dd, J=0.9, 2.6 Hz, 1H), 6.74 (ddd, J=0.8, 2.7, 8.5 Hz, 1H), 3.70 (q, J=7.6 Hz, 1H), 1.56 (d, J=7.6 Hz, 3H)

13C NMR (125 MHz, CD₃OD): δ (ppm) 178.2 (C), 152.4 (C), 147.4 (C), 130.0 (C), 115.1 (CH), 111.3 (CH), 111.3 (CH), 39.0 (CH), 15.9 (CH₃) Scent: woody and leather

### (Example 6) Synthesis of 5-propyl-2,3-dihydrobenzofuran

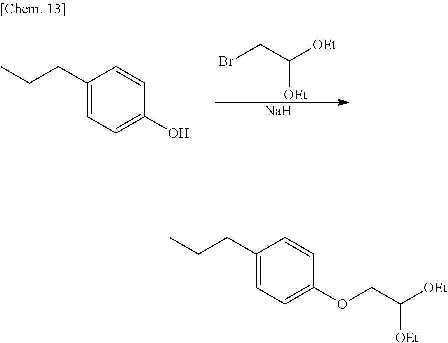

### a) Synthesis of 1-(2,2-diethoxyethoxy)-4-propylbenzene

Under a nitrogen atmosphere, 7.0 g (176 mmol, 1.2 eq.) of sodium hydride and 150 mL of dehydrated DMF were added, and the mixture was cooled to 0° C. To this, a solution of 4-propylphenol (20 g, 147 mmol) was added dropwise in DMF (50 mL). The mixture was warmed to room temperature and stirred for 30 minutes, then 43.6 g (221 mmol, 1.5 eq.) of bromoacetaldehyde diethyl acetal was added thereto, and the mixture was gradually warmed and heated under reflux for 12 hours. The mixture was cooled to room temperature, and 100 mL of a saturated aqueous ammonium chloride solution was slowly added dropwise thereto. To this, 100 mL of ethyl acetate was added, and the aqueous layer was removed. The organic layer was washed with water and rinsed with saturated saline, and dried over sodium sulfate. Sodium sulfate was filtered off, and the solvent was distilled off from the filtrate to give 41 g of a crude product. The crude product was subjected to Vigreux distillation, and the fraction with a column top temperature of 100° C. 105° C. and 120 Pa was collected, to give 29 g (yield: 78%) of 1-(2,2-diethoxyethoxy)-4-propylbenzene.

### Colorless Oil

GC/MS (EI): m/z (%) 252.2 (M⁺, 3), 206.2 (1), 177.1 (1), 161.1 (4), 149.1 (4), 135.1 (1), 119.1 (3), 107.1 (5), 104.1 (2), 103.2 (23), 91.1 (5), 75.1 (14), 61.1 (1), 47.1 (7)

1H NMR (500 MHz, CDCl₃): δ (ppm) 7.07 (d, J=8.6 Hz, 2H), 6.84 (dt, J=2.9, 8.6 Hz, 2H), 4.83 (t, J=5.2 Hz, 1H), 3.99 (d, J=5.2 Hz, 2H), 3.76 (dq, J=7.1, 9.4 Hz, 2H), 3.63 (dq, J=7.1, 9.4 Hz, 2H), 2.52 (t, J=7.5 Hz, 2H), 1.59 (sext, J=7.5 Hz, 2H), 1.24 (t, J=7.1 Hz, 6H), 0.92 (t, J=7.5 Hz, 3H)

13C NMR (125 MHz, CDCl₃): δ (ppm) 156.7 (C), 135.2 (C), 129.3 (CH), 114.4 (CH), 100.5 (CH), 68.6 (CH₂), 62.5 (CH₂), 37, 1 (CH₂), 24.7 (CH₂), 15.3 (CH₃), 13.7 (CH₃)

### b) Synthesis of 5-propylbenzofuran

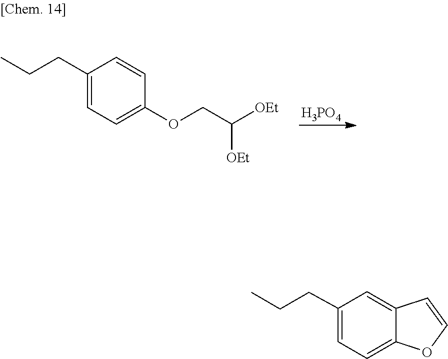

Under a nitrogen atmosphere, 20 g (79.3 mmol) of 1-(2,2-diethoxyethoxy)-4-propylbenzene, 100 mL of toluene, and 16 g (80 wt %) of 70% phosphoric acid were added, and the mixture was heated to reflux for 18 hours. The mixture was cooled to room temperature, and the reaction was stopped by adding 200 mL of a 10% aqueous sodium carbonate solution. The aqueous layer was removed, and the organic layer was washed with water and rinsed with saturated saline, and dried over sodium sulfate. Sodium sulfate was filtered off, and the solvent was distilled off from the filtrate to give 13 g of a crude product. The crude product was subjected to column chromatography to give 8.5 g (yield: 63%) of 5-propylbenzofuran. Further, Vigreux distillation was performed to give 8 g of a fraction at 70° C. and 390 Pa.

GC/MS (EI): m/z (%) 161.1 (M⁺+1, 4), 160.1 (M⁺, 31), 133.1 (1), 132.1 (14), 131.1 (87), 129.1 (1), 128.1 (2), 118.1 (3), 115.1 (4), 103.1 (2), 102.1 (4), 91.1 (2), 89.1 (2), 78.1 (1), 77.1 (12), 76.1 (1), 75.0 (1), 63.1 (3), 51.1 (3), 39.1 (1)

### Colorless Oil

1H NMR (500 MHz, CDCl₃): δ (ppm) 7.58 (d, J=2.2 Hz, 1H), 7.40 (d, J=8.4 Hz, 1H), 7.38 (br.S, 1H), 7.11 (d, J=1.8, 8.4 Hz, 1H), 6.70 (dd, J=0.9, 2.1 Hz, 1H), 2.67 (t, J=7.5 Hz, 2H), 1.67 (sext, J=7.5 Hz, 2H), 0.95 (t, J=7.4 Hz, 3H)

13C NMR (125 MHz, CDCl₃): δ (ppm) 153.5 (C), 145.0 (CH), 137.1 (C), 127.4 (C), 125.0 (CH), 120.4 (CH), 110.9 (CH), 106.4 (CH), 38.0 (CH₂), 25.2 (CH₂), 13.8 (CH₃)

Scent: floral, fruity, and spearmint

### c) Synthesis of 5-propyl-2,3-dihydrobenzofuran

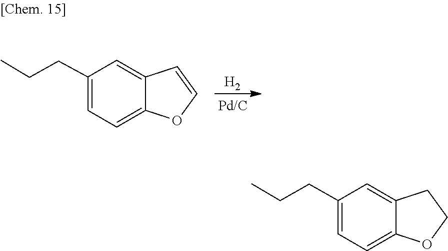

Into a 100 mL autoclave, 2 g (12.5 mmol) of 5-propylbenzofuran, 0.1 g (5 wt %) of 5% Pd/C, and 16 mL of ethyl acetate were added, and the inside of the autoclave was substituted with nitrogen. Thereafter, the inside of the autoclave was substituted with hydrogen three times, 0.7 MPa of hydrogen was sealed in, and the mixture was vigorously stirred at 80° C. for 1 hour. Hydrogen was purged, the catalyst was removed by filtration through Celite, and the solvent was distilled off to give 2.1 g of a crude product. Purification was performed by column chromatography, to give 1.6 g (yield: 80%) of 5-propyl-2,3-dihydrobenzofuran.

### Colorless Oil

GC/MS (EI): m/z (%) 162.1 (M⁺, 11), 134.1 (5), 132.2 (26), 115.1 (1), 105.1 (2), 103.1 (2), 91.1 (2), 79.1 (2), 77.1 (3), 51.1 (1)

1H NMR (500 MHz, CDCl₃): δ (ppm) 7.01 (s, 1H), 6.90 (d, J=8.1 Hz, 1H), 6.69 (d, J=8.1 Hz, 1H), 4.53 (t, J=8.7 Hz, 2H), 3.17 (t, J=8.7 Hz, 2H), 2.51 (t, J=7.5 Hz, 2H), 2.51 (sext, J=7.6 Hz, 2H), 0.93 (t, J=7.3 Hz, 3H)

13C NMR (125 MHz, CDCl₃): δ (ppm) 158.1 (C), 134.7 (C), 127.7 (CH), 126.8 (C), 124.9 (CH), 108.8 (CH), 71.1 (CH₂), 37.5 (CH₂), 29.8 (CH₂), 25.0 (CH₂), 13.8 (CH₃)

Scent: anis, basil, and woody

### (Example 7): Scent Comparison

A panel of 10 trained individuals compared the quality and the intensity of the scent of each of compounds in Example 1 (3-ethylbenzofuran-5-ol), Example 2 (2,3-dimethylbenzofuran-5-ol), and Example 3 (2-ethyl-3-methylbenzofuran-5-ol: 3-ethyl-2-methylbenzofuran-5-ol=55:45), and each of compounds of 3-methylbenzofuran-5-ol (synthesized as described in US2015/111810A1) as Comparative Example 1, 3-propylbenzofuran-5-ol (synthesized as described in Tetrahedron, 1990, 46, 8153-8160) as Comparative Example 2, and 3-isopropoxybenzofuran-5-ol (synthesized as described in US2015/111810A1) as Comparative Example 3.

The numerical value of the scent intensity ranges from 10 (strong) to 1 (weak).

TABLE 1

| | Compound | Scent | Intensity |
|---|---|---|---|
| Exam- ple 1 | 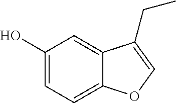 | Leather, phenolic, animalic, cedar- wood, soft | 10 |
| Exam- ple 2 | 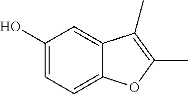 | Leather, animalic, moss, earthy, woody | 7 |
| Exam- ple 3 | 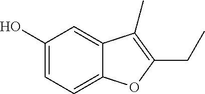 | Woody, leather, moss, animalic, oud, clean | 9 |
| | (55%) + | | |
| | 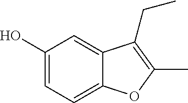 | | |
| | (45%) | | |
| Com- par- ative Exam- ple 1 | 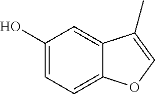 | Leather, phenolic, animalic, earthy | 6 |
| Com- par- ative Exam- ple 2 | 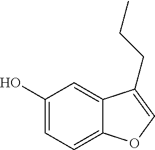 | Phenolic, woody, creamy | 3 |
| Com- par- ative Exam- ple 3 | 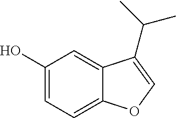 | Phenolic, balsamic, creamy, woody, cedar- wood | 4 |

It was revealed that the compounds in Examples 1 to 3 have a leather-like or moss-like scent, and clearly more excellent intensity of scent than the compounds in Comparative Examples 1 to 3.

It was also revealed that in the case of comparing the compound in Example 1 with the compounds in Comparative Examples 1 to 3, it is not as simple as just extending the alkyl carbon at the R1 position (i.e., the 3-position on the benzofuran ring), but the scent intensity is maximized when the alkyl carbon has an appropriate length (ethyl group). In addition, it was also revealed that when the substituent group is changed from an ethyl group to an n-propyl group or an i-propyl group, a preferred leather-like scent property almost disappears, and the scent changes to a lesser value of phenolic and woody scent.

### (Example 8) Fragrance Composition

According to the formulation in Table 2 below, a fragrance composition was prepared using the compound synthesized in Example 1 above.

TABLE 2

| Formulation | (part by mass) |
|---|---|
| Allyl caproate | 14 |
| L-citronellyl nitrile | 6 |
| CYCLAPROP (registered trademark) | 30 |
| α-damascone | 12 |
| Ethyl 2-methylbutyrate | 10 |
| Ethyl methylphenylglycidate | 8 |
| Eugenol | 2 |
| FRUITATE (registered trademark) | 10 |
| Geranyl acetate | 16 |
| HEDIONE (registered trademark) | 100 |
| HELIOBOUQUET (registered trademark) | 6 |
| Cis-3-hexenol | 2 |
| Hexyl acetate | 30 |
| Hexyl cinnamic aldehyde | 70 |
| Hexyl salicylate | 50 |
| HINDINOL (registered trademark) | 8 |
| Cis-jasmon | 1 |
| Linalyl acetate | 10 |
| Orange oil | 50 |
| L-ORANTHA SUPER (registered trademark) | 6 |
| 1.0% DPG solution | |
| ORBITONE (registered trademark) | 20 |
| PEONILE (registered trademark) | 14 |
| PHENOXANOL (registered trademark) | 20 |
| Raspberry ketone | 36 |
| ROSYRANE SUPER 1.0% DPG solution | 4 |
| Tetrahydrolinalool | 100 |
| TRIPLAL (registered trademark) | 4 |
| γ-undecalactone | 20 |
| VERDOX | 60 |
| Yara Yara | 2 |
| Dipropylene glycol | 209 |
| Compound in Example 1 | 70 |
| Total | 1000 |

### (Example 9) Shampoo

According to the formulation in Table 3 below, a shampoo (100 g) to which 1.0 mass % of the fragrance composition in Example 8 was added was prepared.

TABLE 3

| Formulation (Component) | (Blending amount g) |
|---|---|
| Sodium polyoxyethylene lauryl ether sulfate | 14.00 |
| Lauramidopropyl betaine | 4.00 |
| Coconut oil fatty acid diethanolamide | 3.00 |
| Cationic cellulose | 0.50 |
| Ethylene glycol distearate | 1.00 |
| Ethyl paraoxybenzoate | 0.25 |
| Citric acid | Appropriate amount |
| Fragrance composition in Example 8 | 1.00 |
| Purified water | Remainder |
| Total | 100.00 |

Although the present invention has been described in detail and with reference to specific embodiments, it will be apparent to those skilled in the art that various changes and modifications can be made without departing from the spirit and scope of the present invention. This application is based on U.S. Patent Application No. 63/421,783 filed on Nov. 2, 2022, the contents of which are incorporated herein by reference.

1. A fragrance composition comprising a compound represented by the following general formula (1): 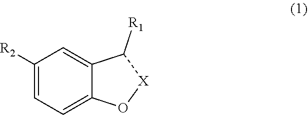
wherein R₁ represents a hydrogen atom, a methyl group, or an ethyl group,
R₂ represents a hydroxy group, an alkyl group having 1 to 5 carbon atoms, or an alkoxy group having 1 to 5 carbon atoms, and
the dotted line represents a single bond or a double bond, and
in a case where the dotted line is a single bond, X represents a methylene group or a carbonyl group, and
in a case where the dotted line is a double bond, X represents C—R₃ where R3 represents a hydrogen atom, a methyl group, or an ethyl group, where R₃ represents a methyl group or an ethyl group in a case where R₁ is a methyl group.

2. The fragrance composition according to claim 1, wherein the compound represented by the general formula (1) includes one or more compound represented by the following formulas:

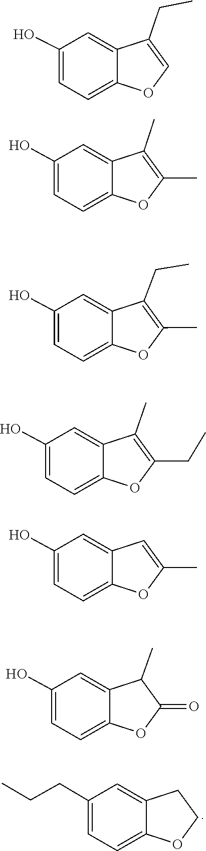

3. A consumer product comprising the fragrance composition according to claim 1,
wherein the consumer product is selected from a fragrance product, a cosmetic, a toiletry product, an air care product, daily necessities, household goods, an oral care product, a detergent, a softener, a quasi-drug, a beverage, or a food.

4. A consumer product comprising the fragrance composition according to claim 2,
wherein the consumer product is selected from a fragrance product, a cosmetic, a toiletry product, an air care product, daily necessities, household goods, an oral care product, a detergent, a softener, a quasi-drug, a beverage, and food.
